(19) United States
(12) Patent Application Publication
(10) Pub. No.: US 2020/0155775 A1
(43) Pub. Date: May 21, 2020

(54) FLUID PRODUCT DISPENSING DEVICE

(71) Applicant: APTAR FRANCE SAS, LE NEUBOURG (FR)

(72) Inventors: Frank KEPPNER, Albstadt (DE); Joachim KORNER, Uhldingen-Muhlhofen (DE)

(73) Assignee: APTAR FRANCE SAS, LE NEUBOURG (FR)

(21) Appl. No.: 16/635,391

(22) PCT Filed: Aug. 2, 2018
(86) PCT No.: PCT/FR2018/051993

§ 371 Date: Jan. 30, 2020

(30) Foreign Application Priority Data
Aug. 3, 2017 (FR) 1757460

(51) Int. Cl. A61M 15/00; A61M 11/02

(52) U.S. Cl. CPC A61M 15/009; A61M 11/02; A61M 15/008; A61M 15/0025

(57) ABSTRACT

A fluid dispenser device having a body (10) provided with a mouthpiece (15); a fluid reservoir (20) containing a fluid and a propellant gas; and a metering valve (30) including a valve member (32). The valve member is received in a valve well (50) secured to main body (10). The reservoir (20) is mounted in the body (10) to slide between a rest position in which the metering valve is closed and a dispensing position it is open to dispense a dose of fluid. The device includes an actuation sensor, an orientation sensor and/or a movement sensor (500); an electronic dose counter including a screen (1400) displaying the number of doses; and a signal-transmitter (1500) for communicating information relating to the actuations of the device.

The present invention relates to a fluid dispenser device, and more particularly to an inhaler device of the aerosol type.

Inhaler devices are well known. In particular, “Metered Dose Inhaler” (MDI) or “pressurized Metered Dose Inhaler” (pMDI) devices generally comprise a body slidably receiving a reservoir containing both fluid to be dispensed and also a propellant gas. A metering valve is mounted on said reservoir for selectively dispensing the fluid during actuation.

Several problems can occur with devices of that type.

Thus, actuation must be performed in the appropriate position, with the reservoir arranged above the valve, so as to guarantee reliable filling of the metering chamber when the valve returns to its rest position, after actuation. Thus, by way of example, if the device is actuated by a user in a prone position, the device is not properly orientated, and there is a risk of the dose being incomplete during the next actuation.

Another problem relates to the uniformity and to the reproducibility of the dose on each actuation, in particular after a long period of storage. Generally, the operating instructions of the device call for shaking the device before actuation, but nothing prevents the user from actuating it without shaking, nor reminds the user that the device must be shaken before actuation. Thus, there is a risk that the device is actuated without being shaken beforehand.

Another problem inherent to devices incorporating electronic components relates to the lifetime of the power supply, generally a battery. Optimizing power management is thus important with that type of device.

Still another problem relates to tracking successive actuations of the device remotely. Although connected type devices exist that make it possible to inform a third party that the device has been actuated, it is not possible at present to identify the identity of the user reliably, nor whether the dose has been inhaled in the required proper conditions.

An object of the present invention is to provide a fluid dispenser device that does not have the above-mentioned drawbacks.

Another object of the present invention is to provide a fluid dispenser device having operational reliability that is improved.

Another object of the present invention is to provide a fluid dispenser device that improves the reproducibility of the dose to be dispensed on each actuation.

Another object of the present invention is to provide a fluid dispenser device that reduces the risks of the electronic components malfunctioning.

Another object of the present invention is to provide a fluid dispenser device that makes it possible, at least in part, to identify remotely the user of the device and the quality of the dose as actually dispensed.

Another object of the present invention is to provide a fluid dispenser device that is simple and inexpensive to manufacture and to assemble.

The present invention thus provides a fluid dispenser device comprising: a main body provided with a mouthpiece; a fluid reservoir containing both a fluid and also a propellant gas; and a metering valve including a valve member, which metering valve is assembled on said reservoir for selectively dispensing the fluid; said valve member being received in a valve well that is secured to said main body, and said reservoir being mounted in said main body to slide between a rest position in which said metering valve is closed, and a dispensing position in which said metering valve is open so as to dispense a dose of fluid through said valve member towards said mouthpiece, said device further comprising:
at least one actuation sensor for detecting the actuation of the device and/or the dispensing of a dose of fluid; and
at least one orientation sensor and/or movement sensor for detecting the orientation of the device and/or the movements of the device;
an electronic dose counter including a screen displaying the number of doses that have been dispensed or that remain to be dispensed; and
signal-transmitter means for communicating, in particular for communicating remotely, information relating to the actuations of the device.

Advantageously, said orientation sensor comprises an accelerometer.

Advantageously, said movement sensor comprises an accelerometer.

Advantageously, said at least one accelerometer detects movements of the user's arm and/or hand during actuation.

In a first advantageous variant, said actuation sensor is arranged in the fluid expulsion path.

Advantageously, said actuation sensor is a membrane that is arranged in the valve well.

In a second advantageous variant, said actuation sensor is arranged outside the fluid expulsion path.

Advantageously, said actuation sensor is a sound sensor, in particular for detecting the sound generated while dispensing the fluid.

Advantageously, said actuation sensor is a movement sensor, preferably comprising an accelerometer.

Advantageously, said sensors are arranged on the main body.

Advantageously, said sensors are arranged on the reservoir.

Advantageously, said device further comprises a contact sensor or switch, in particular so as to cause at least one sensor to pass from a “standby” mode to a “wakeup” mode.

Advantageously, said actuation sensor actuates said dose counter.

These and other characteristics and advantages appear more clearly from the following detailed description, given by way of non-limiting example, and with reference to the accompanying drawings, in which:

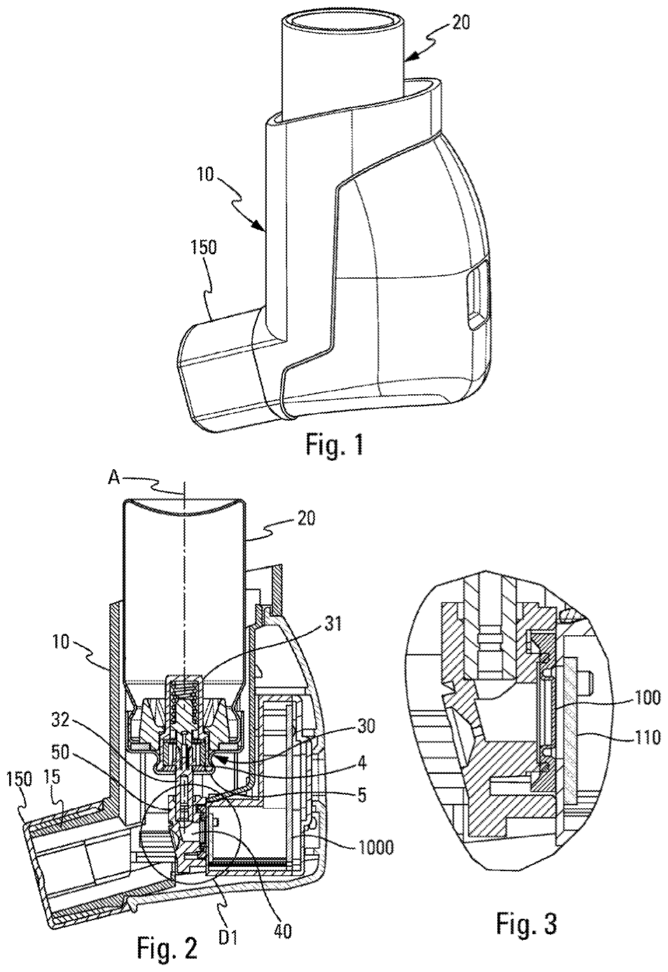

FIG. 1 is a diagrammatic perspective view of a fluid dispenser device, in a first advantageous embodiment, in its rest position;

FIG. 2 is a diagrammatic section view of the FIG. 1 device;

FIG. 3 is a larger-scale view of a detail D1 of FIG. 2;

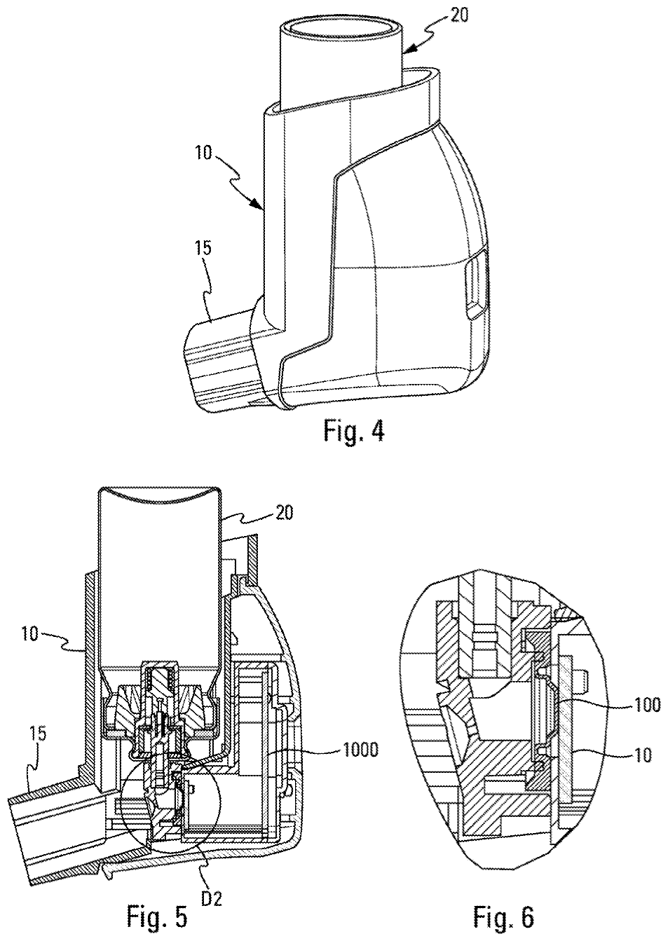

FIG. 4 is a view similar to the view in FIG. 1, in the actuated position;

FIG. 5 is a diagrammatic section view of the FIG. 4 device;

FIG. 6 is a larger-scale view of a detail D2 of FIG. 5;

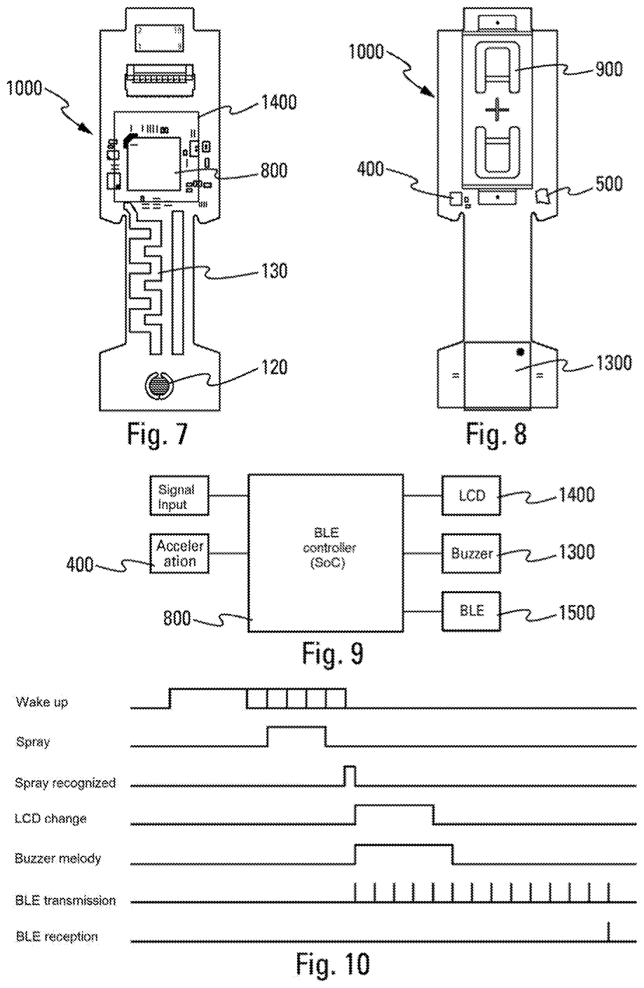

FIG. 7 is a section view as seen from above, showing an example of a printed circuit used in the device in FIGS. 1 to 6;

FIG. 8 is a view similar to the view in FIG. 7, as seen from below;

FIG. 9 is a diagrammatic view of an example of a system on a chip used in the device in FIGS. 1 to 6;

FIG. 10 is a timing chart showing an example of a sequence of signals generated by the device in FIGS. 1 to 6;

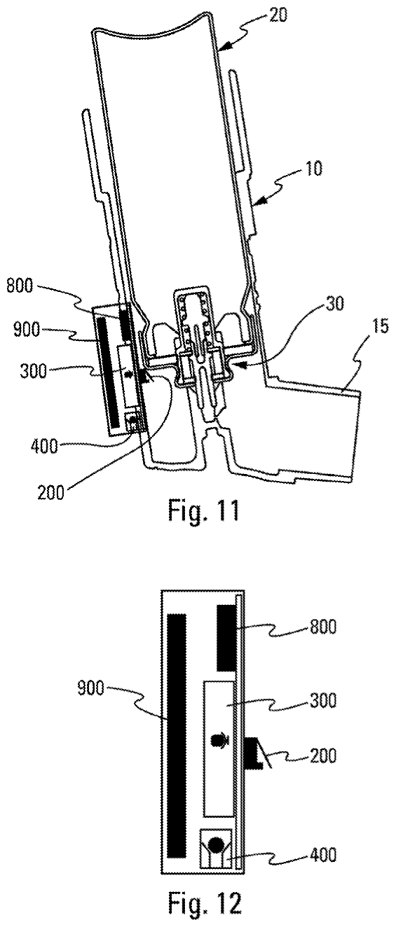

FIG. 11 is a diagrammatic perspective view of a fluid dispenser device, in a second advantageous embodiment, in its rest position;

FIG. 12 is a larger-scale view of the FIG. 11 electronic components;

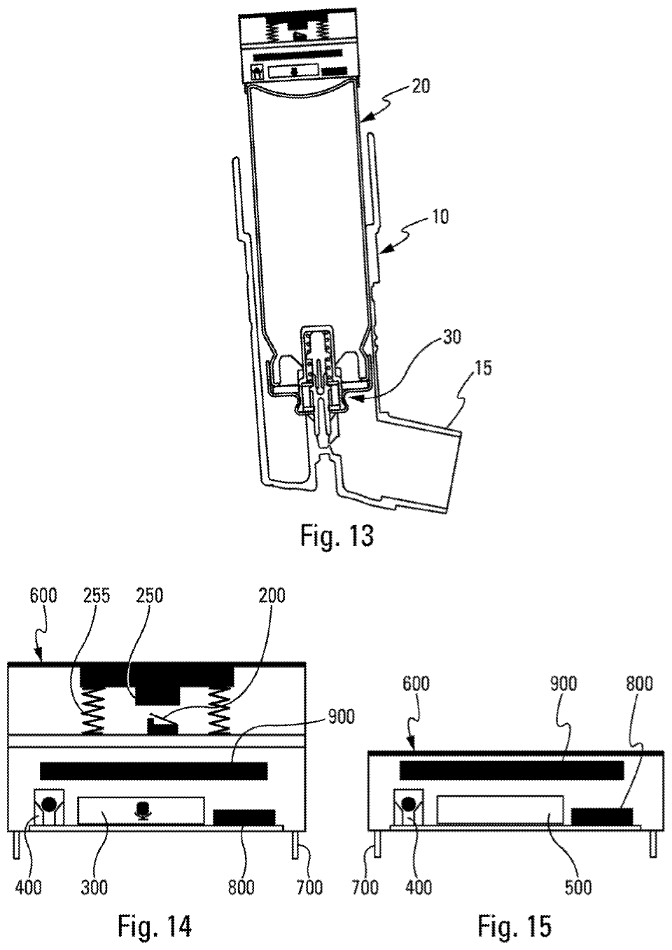

FIG. 13 is a diagrammatic perspective view of a fluid dispenser device, in a third advantageous embodiment, in its rest position;

FIG. 14 is a larger-scale view of the FIG. 13 electronic components; and

FIG. 15 is a variant of FIG. 14.

In the description, the terms “top”, “bottom”, “upwards”, and “downwards” are relative to the upright position of the device shown in FIGS. 1, 2, 4, and 5. The terms “axial” and “radial” are relative to the vertical central axis A shown in particular in FIG. 2. The terms “proximal” and “distal” are relative to the mouthpiece.

The invention applies to inhaler devices of the aerosol-valve type for oral dispensing, as described in greater detail below, and generally referred to by the terms “Metered Dose Inhaler” (MDI) or “pressurized Metered Dose Inhaler (pMDI).

The figures show two advantageous embodiments of the invention, but naturally one or more of the component parts described below could be made in some other way, while providing functions that are similar or identical.

With reference to the figures, showing various advantageous embodiments of the invention, the device includes a main body 10 provided with a mouthpiece 15. The mouthpiece 15 defines a dispenser orifice through which the user inhales while the device is being used. The mouthpiece 15 may be made integrally with the body 10, as in FIGS. 1, 5, 11, and 13, but it could also be formed on a bottom body portion that is fastened to said main body 10. A removable protective cap 150 may be provided on said mouthpiece 15, in particular while it is being stored, that the user removes before use. FIGS. 1 and 2 show such a protective cap 150 that could be of any shape.

The main body 10 contains a reservoir 20 that contains both the fluid to be dispensed and also a propellant gas, such as a gas of the hydrofluoroalkane (HFA) type, a metering valve 30 being mounted on said reservoir 20 for selectively dispensing the fluid.

The metering valve 30 comprises a valve body 31, and a valve member 32 that, during actuation, is axially movable relative to said valve body 31, and thus relative to said reservoir 20. The metering valve 30 can be of any appropriate type. It may be fastened to the reservoir 20 via a fastener element 5, preferably a crimped cap, preferably with a neck gasket 4 interposed therebetween.

The outlet orifice of the valve member 32 of said metering valve 30 is connected via a channel 40 to said mouthpiece 15 through which the user inhales the fluid to be dispensed. In known manner, said valve member 32 is received in a valve well 50 that defines said channel 40, at least in part. The valve well 50 may be formed integrally with the main body 10 (or with a bottom body portion as mentioned above), but it may also be formed by a separate part that comes to be fastened in said main body 10.

The reservoir 20 is mounted in the main body 10 to slide axially between a rest position in which the metering valve 30 is closed, and a dispensing position in which the metering valve 30 is open so as to dispense a dose of fluid through the valve member 32 towards said mouthpiece 15.

In the invention, the device includes electronic modules with various sensors.

At least one actuation sensor may be provided for detecting the actuation of the device. The actuation sensor may be a membrane sensor 100, as shown in FIGS. 1 to 6, that detects the dispensing of the dose of fluid, e.g. in the valve well 50. As shown in particular in FIGS. 3 and 6, in the rest position and before use, the membrane 100 is passive and no signal is created. During actuation, the membrane 100 deforms and thus creates an event by coming into contact with an appropriate contact plate 110 for generating a signal. By way of example, the signal may be used to change the display on an LCD screen of an electronic counter provided in the device.

In a variant, the actuation sensor could detect the movements of the reservoir 20, e.g. by means of an appropriate contact sensor or switch 200, shown in particular in FIGS. 11, 12, 13, and 14. The movement of the reservoir 20 may be detected from the start of the actuation stroke. The contact sensor 200 may thus be used to cause the various electronic modules to pass from an energy-saving “standby” mode to a “wakeup” or “ready to be actuated” mode.

Actuation of the device may be detected in other ways, e.g. by detecting the movement of the valve member 32 of the metering valve 30 relative to the valve body 31.

Another variant of an actuation sensor is shown in FIGS. 11 to 14. In these variants, the actuation sensor is a sound sensor 300 that is adapted to detect the sound produced by the actuation of the device. Preferably, it detects the sound generated by the dose being expelled from the valve 30. In a variant, it is also possible to detect other sounds that are characteristic of the actuation of the device and/or of the dispensing of a dose, such as the sound generated by the movement of the reservoir 20 in the body 10, or the sound generated by the movement of the valve member 32 in the valve body 31, for example.

At least one orientation sensor 400 may be provided for detecting the orientation of the device, in particular while it is being actuated. The orientation sensor 400 comprises an accelerometer. Such an orientation sensor 400 makes it possible in particular to detect whether the device is properly orientated, in particular so as to guarantee effective filling of the valve chamber when said valve chamber returns to its rest position after each actuation. The orientation sensor 400 also makes it possible to detect whether the user shakes the device before use, as is generally recommended in the operating instructions.

At least one movement sensor 500 may be provided for detecting movements of the user's arm or hand during actuation. The movement sensor may comprise an accelerometer. Such a movement sensor 500 may make it possible in particular to identify a user from the actuation movement, since each user has a specific movement profile. The movement sensor 500 also makes it possible to detect whether the user shakes the device before use, as is generally recommended in the operating instructions. The movement sensor 500 also makes it possible to detect actuation of the device, as a replacement for the sound sensor 400, as in the variant in FIG. 15.

The orientation sensor 400 or the movement sensor 500 could also be used to cause the various electronic modules to pass from an energy saving “standby” mode to a “wakeup” or “ready to be actuated” mode.

The device may include recorder means for recording the date and the time, in particular for timestamping each actuation of the device.

A GPS sensor may be provided, in particular for geolocating each actuation of the device.

The device includes storage means for storing at least some of the information detected by the various electronic modules, a microcontroller 800 for controlling the various electronic modules, and power supply means 900, such as an optionally rechargeable battery.

The device may include a loud speaker or buzzer 1300. By way of example, said loud speaker or buzzer may indicate to the user that the device has been actuated properly and/or that the dose has been dispensed properly. It may also serve as a warning, e.g. in the event of a low battery, or to indicate to the user that the device must be actuated.

The device may also include an electronic dose counter with a screen 1400 displaying the number of doses that have been dispensed or that remain to be dispensed. The counter may be connected to the actuation sensor, in particular the membrane sensor 100 in FIGS. 1 to 6 or the contact sensor 200 in FIGS. 11 and 12. Documents WO 2015/150029 and WO 2015/010932 describe counters that function with a membrane sensor 100.

The device may also include signal-transmitter means 1500 for communicating information relating to the actuations of the device. In particular, the body 10 may include a signal-transmitter module, for communicating remotely with any receptors. Appropriate power supply means are advantageously provided.

Advantageously, said signal-transmitter means 1500 are active or passive wireless means, such as Bluetooth (BLE), Wifi, NFC, RFID, etc., for sending information to a separate peripheral. A wired connection associated with a base is also possible. The data stored may thus be transferred automatically or on demand.

FIGS. 7 to 10 show the electronic modules of the device in FIGS. 1 to 6. The modules are formed on a printed circuit card 1000 that may support some or all of the above-described elements. It should be observed that the example in FIGS. 7 to 10 is not limiting, and that other combinations of modules would be possible.

In this example, the signal generated during actuation by the membrane 100 is received on the card 1000 at an inlet 120. An antenna 130 then transmits the signal to the controller 800 that controls one or more peripherals, such as the screen 1400 and/or the loud speaker 1300. The orientation sensor 400 and/or the movement sensor 500 are also connected to said controller 800.

FIG. 10 shows a possible sequence of signals generated during actuation.

In the embodiment in FIGS. 1 to 6, actuation is detected by means of the membrane 100 arranged in the fluid expulsion path. Specifically, this comprises detecting dose dispensing proper.

In some circumstances, it may not be desirable to modify the fluid expulsion path. In such circumstances, the sensors need to be positioned outside said path, as shown by the embodiments in FIGS. 11 to 15.

In the example in FIGS. 11 and 12, the electronic modules are arranged laterally in the body 10.

In this example, the device includes a switch 200, a sound sensor 300, and an orientation sensor 400.

When the user takes hold of the device and presses on the reservoir 20 so as to actuate it, the switch 200 is actuated, e.g. by an element secured to the reservoir, in particular the cap 5, generating a signal. The signal may be used to activate or “wake up” the other sensors.

The sound detected by the sound sensor 300 is preferably the sound generated by the spray, i.e. the fluid being expelled from the valve member. In a variant, provision could also be made for a movement sensor 500, e.g. as a replacement for the sound sensor 300.

The acceleration signal of the orientation sensor 400 and/or of the movement sensor 500 is mainly generated by the back-pressure created during spraying.

In the examples in FIGS. 13 to 15, the electronic modules are arranged on the end wall of the reservoir 20.

FIGS. 13 and 14 show a first variant in which the device includes a switch 200, a sound sensor 300, an orientation sensor 400, a capacitive sensor 600, and a position sensor 700.

The capacitive sensor 600 is provided on a distal end surface of the body receiving the electronic modules, so as to detect the pressure of the user's fingers.

The position sensor 700 is provided so as to detect that the body receiving the electronic modules is fastened to the reservoir 20.

When the user takes hold of the apparatus, the capacitive sensor 600 detects the user, and the orientation sensor 400 is activated so as to detect whether the user shakes the device. If not, a signal or message can be transmitted to the user via the loud speaker or the screen.

In this example, the switch 200 is provided in a user interface 250 that is spring-loaded by a spring 255 having a resistance that is less than the resistance of the valve 30, so that the switch 200 is actuated before the reservoir 20 is moved in the body 10.

When the patient actuates the device, the switch 200 is thus actuated first, generating a signal that activates or “wakes up” the sound sensor 300.

Then, the valve is actuated and a dose of fluid is dispensed.

When the sound sensor 300 picks up the sound corresponding to a spray, the dose counter is actuated.

During the return stroke, after spraying, the orientation sensor 400 verifies whether the orientation of the device is correct. If it is not, the user can be informed, and the user can be requested in particular to perform a priming actuation so as to avoid the dose being incomplete on the next actuation.

At the end of actuation, the device, and in particular the electronic modules, return(s) to standby mode, e.g. when the capacitive sensor 600 is no longer activated.

FIG. 15 shows another variant, without the user interface 250, without the switch 200, and with a movement sensor 500 as a replacement for the sound sensor. The device thus includes an orientation sensor 400, a movement sensor 500, an (optional) capacitive sensor 600, and a position sensor 700.

When the user takes hold of the apparatus, the capacitive sensor 600, if it is used, detects the user, and the orientation sensor 400 is activated so as to detect whether the user shakes the device. If not, a signal or message can be transmitted to the user via the loud speaker or the screen.

When the patient actuates the device, the movement sensor 500 detects the movement of the reservoir and generates a corresponding signal. Depending on the settings, the movement sensor can be actuated only after a certain predefined stroke of the reservoir 20 in the body 10.

During the return stroke, after spraying, the orientation sensor 400 verifies whether the orientation of the device is correct. If it is not, the user can be informed, and the user can be requested in particular to perform a priming actuation so as to avoid the dose being incomplete on the next actuation.

Once the movement sensor 500 no longer detects a signal for a defined period of time, the electronic modules may be returned to standby mode. When using the capacitive sensor 600, said capacitive sensor may also be used for this purpose.

In the embodiment in FIG. 15, it should be observed that, depending on the capabilities of the movement sensor 500, the presence of the orientation sensor may turn out to be unnecessary, and said orientation sensor may be omitted. Specifically, when the movement sensor comprises an accelerometer, said accelerometer may perform both functions, detection of movement and detection of orientation.

The present invention is described above with reference to advantageous embodiments, but naturally any modification could be applied thereto by a person skilled in the art, without going beyond the ambit of the present invention, as defined by the accompanying claims.

1.-13. (canceled)

14. A fluid dispenser device comprising: a main body provided with a mouthpiece; a fluid reservoir containing both a fluid and also a propellant gas; and a metering valve including a valve member, which metering valve is assembled on said reservoir for selectively dispensing the fluid through a fluid expulsion path that opens out into said mouthpiece; said valve member being received in a valve well that is secured to said main body, and said reservoir being mounted in said main body to slide between a rest position in which said metering valve is closed, and a dispensing position in which said metering valve is open so as to dispense a dose of fluid through said valve member towards said mouthpiece, said device further comprising:
at least one actuation sensor for detecting the actuation of the device and/or the dispensing of a dose of fluid; and
at least one orientation sensor and/or movement sensor for detecting the orientation of the device and/or the movements of the device;
an electronic dose counter including a screen displaying the number of doses that have been dispensed or that remain to be dispensed; and
signal-transmitter means for communicating, in particular for communicating remotely, information relating to the actuations of the device;
wherein said orientation sensor and/or said movement sensor comprises an accelerometer, said at least one accelerometer detecting movements of the user's arm and/or hand during actuation, so as to identify the user from the actuation movement.

15. A device according to claim 14, wherein said actuation sensor is arranged in the fluid expulsion path.

16. A device according to claim 15, wherein said actuation sensor is a membrane that is arranged in the valve well.

17. A device according to claim 14, wherein said actuation sensor is arranged outside the fluid expulsion path.

18. A device according to claim 17, wherein said actuation sensor is a sound sensor, in particular for detecting the sound generated while dispensing the fluid.

19. A device according to claim 17, wherein said actuation sensor is a movement sensor, preferably comprising an accelerometer.

20. A device according to claim 17, wherein said sensors are arranged on the main body.

21. A device according to claim 17, wherein said sensors are arranged on the reservoir.

22. A device according to claim 14, further comprising a contact sensor or switch, in particular so as to cause at least one sensor to pass from a “standby” mode to a “wakeup” mode.

23. A device according to claim 14, wherein said actuation sensor actuates said dose counter.
